(19) United States
(12) Patent Application Publication
(10) Pub. No.: US 2010/0217034 A1
(43) Pub. Date: Aug. 26, 2010

(54) Process for the Preparation of Fesoterodine

(75) Inventors: Kishore Charugundla, Andhra Pradesh (IN); Udhaya Kumar, Pondecherry (IN); Rajendra Suryabhan Patil, Tal-Skah Dist-Thane (IN); Praveen Kumar Neela, Andhra Pradesh (IN); Nitin Sharadchandra Pradhan, Maharashtra (IN); Jon Valgeirsson, Hafnarfjordur (IS)

Correspondence Address: CANTOR COLBURN, LLP, 20 Church Street, 22nd Floor, Hartford, CT, 06103, US

(73) Assignee: ACTAVIS GROUP PTC EHF, 220 Hafnarfjordur (IS)

(21) Appl. No.: 12/678,845

(22) PCT Filed: Sep. 22, 2008
(86) PCT No.: PCT/IB08/03098

§ 371 Date: May 3, 2010

(30) Foreign Application Priority Data
Sep. 21, 2007 (IN) 2129/CHE/2007
Dec. 28, 2007 (IN) 3137/CHE/2007

(51) Int. Cl. C07C 69/02; C07C 211/52

(52) U.S. Cl. 560/250; 564/442

(57) ABSTRACT

Disclosed herein is an improved, commercially viable and industrially advantageous process for the preparation of Fesoterodine or a pharmaceutically acceptable salt thereof in high yield and purity. Disclosed also herein is an improved and industrially advantageous optical resolution method of racemic (±)-N,N-Diisopropyl-3-(2-benzyloxy-5-bromophenyl)-3-phenylpropylamine and use thereof for the preparation of Fesoterodine.

### CROSS-REFERENCE TO RELATED APPLICATION

This application claims the benefit of priority to Indian provisional application Nos. 2129/CHE/2007, filed, on Sep. 21, 2007, and 3137/CHE/2007, filed on Dec. 28, 2007, which are incorporated herein by reference.

### FIELD OF THE INVENTION

Disclosed herein is an improved, commercially viable and industrially advantageous process for the preparation of Fesoterodine or a pharmaceutically acceptable salt thereof in high yield and purity. Disclosed also herein is an improved and industrially advantageous optical resolution method of racemic (±)-N,N-Diisopropyl-3-(2-benzyloxy-5-bromophenyl)-3-phenylpropylamine and use thereof for the preparation of Fesoterodine.

### BACKGROUND OF THE INVENTION

U.S. Pat. No. 6,713,464 B1 disclosed a variety of 3,3-diphenylpropylamine derivatives, processes for their preparation, pharmaceutical compositions in which they are present and method of use thereof. These compounds are anti-muscarinic agents with superior pharmacokinetic properties compared to existing drugs such as oxybutynin and tolterodine and useful in the treatment of urinary incontinence, gastrointestinal hyperactivity (irritable bowel syndrome) and other smooth muscle contractile conditions. Among them, Fesoterodine, chemically 2-[(1R)-3-[bis(1-methylethyl)amino]-1-phenylpropyl]-4-hydroxymethylphenyl isobutyrate is a new, potent and competitive muscarinic antagonist and useful in the potential treatment of urinary incontinence. Fesoterodine is represented by the following structural formula I:

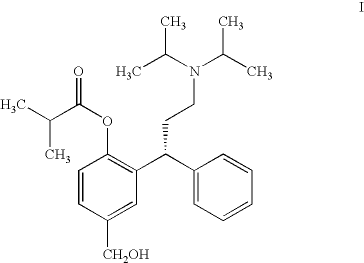

Processes for the preparation of fesoterodine and related compounds, and their pharmaceutically acceptable salts were disclosed in the U.S. Pat. Nos. 6,713,464 B1 and 6,858,650 B1; U.S. Patent Application No. 2006/0270738 and PCT Publication No. WO 2007/138440 A1.

In the preparation of fesoterodine, (R)—N,N-diisopropyl-3-(2-benzyloxy-5-bromophenyl)-3-phenylpropylamine of formula VII(i):

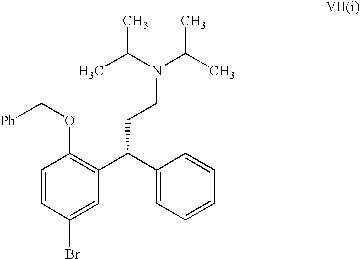

is a key intermediate. A previously known method for the synthesis of intermediate, (R)—N,N-diisopropyl-3-(2-benzyloxy-5-bromophenyl)-3-phenylpropylamine was reported in the U.S. Pat. No. 5,559,269, which involves the resolution of racemic (±)-N,N-diisopropyl-3-(2-benzyloxy-5-bromophenyl)-3-phenylpropylamine of formula VI(i):

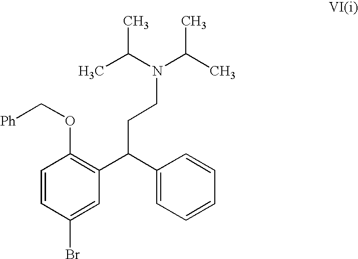

by using L-(+)-tartaric acid as the optically active acid and in the presence ethanol and diethyl ether and subsequent decomposition of the salt.

The main problem associated with this process is that it does not end up with crystallized solid. The (R)-amine compound of formula VII(i) obtained by the process described in the '269 patent does not have satisfactory chiral purity. The process used in the '269 patent also suffers from disadvantages such as low yields of the product and extra purification steps.

The object of the present invention is to provide a commercially useful procedure for obtaining the desired enantiomer of the compound of formula VI(i) separately with a good yield and suitable enantiomeric purity, and its use thereof for the preparation of fesoterodine. Desirable process properties include non-hazardous and environmentally friendly reagents, reduced cost, greater simplicity, increased enantiomeric and chemical purity, and increased yield of the product.

According to the U.S. Pat. No. 6,713,464 B1 (herein after referred to as the '464 patent), fesoterodine was prepared by the reaction of (±)-6-bromo-4-phenylchroman-2-one with benzyl chloride in the presence of sodium iodide and anhydrous potassium carbonate in methanol and acetone to give (±)-3-(2-benzyloxy-5-bromophenyl)-3-phenylpropionic acid methyl ester as a light yellow oil, which by reduction with lithium aluminium hydride in tetrahydrofuran at room temperature (reaction time: 18 hours) to produce (±)-3-(2-benzyloxy-5-bromophenyl)-3-phenylpropan-1-ol, which is then treated with p-toluenesulphonyl chloride in the presence of pyridine in dichloromethane to afford (±)-toluene-4-sulphonic acid 3-(2-benzyloxy-5-bromophenyl)-3-phenylpropyl ester followed by reaction with N,N-diisopropylamine in acetonitrile at reflux temperature (i.e., 75-80° C.) for 97 hours to produce (±)-[3-(2-benzyloxy-5-bromophenyl)-3-phenylpropyl]-diisopropylamine as a brown and viscous syrup, followed by resolution to produce (R)-[3-(2-benzyloxy-5-bromophenyl)-3-phenylpropyl]-diisopropylamine, which is then subjected to Grignard reaction with ethylbromide and magnesium in the presence of solid carbon dioxide in tetrahydrofuran to produce (R)-4-benzyloxy-3-(3-diisopropylamino-1-phenylpropyl)-benzoic acid hydrochloride followed by esterification with methanol in the presence of sulphuric acid to produce (R)-4-benzyloxy-3-(3-diisopropylamino-1-phenylpropyl)-benzoic acid methyl ester, which is then reduced with lithium aluminium hydride (reaction time: 18 hours) to produce (R)-[4-benzyloxy-3-(3-diisopropylamino-1-phenylpropyl)-phenyl]-methanol, which is then subjected to deprotection with Raney-Nickel to produce (R)-2-(3-diisopropylamino-1-phenylpropyl)-4-hydroxymethylphenol followed by condensation with isobutyryl chloride in an inert solvent in the presence of a base to give fesoterodine.

The above process utilizes lithium aluminium hydride as a reducing agent in the reduction reaction and it takes 18 hours for reaction completion. The present inventors surprisingly found that when other metal hydride such as sodium borohydride is used as a reducing agent in presence of lewis acid the reaction will be completed in 2 hours and yields the resulting product in high purity and in good yield.

The above process utilizes pyridine as a base in the tosylation reaction. The present inventors found that when pyridine is used as a base the reaction will not go for completion and takes longer time. When aliphatic organic base such as triethyl amine is used as a base the reaction proceeds for completion.

In the above process, the amination reaction is carried out at reflux temperature i.e. 75-80° C. for 97 hours. This leads some impurity formation and the product isolated as oily mass with 78% yield. The present inventors found that when the amination reaction is carried out in autoclave at 70-140° C. the reaction will be completed in 30 hours and this process yields 90%.

The above prior art process involves the use of methanol in presence of sulfuric acid for esterification reaction. The present inventors found that when the esterfication is carried out in presence of sulfuric acid the reaction will not go for completion. However, the esterification reaction proceeds for completion by using acid chloride such as thionyl chloride in place of sulfuric acid.

Fesoterodine obtained by the process described in the '464 patent is not satisfactory from purity point of view, the yields are very low, and have the following disadvantage and limitations:
i) Expensive and hazardous reagent like Lithium aluminium hydride is difficult to use at commercial scale since it reacts with water, including atmospheric moisture, and the pure material is pyrophoric.
ii) Amination reaction involves 97 hours for completion.
iii) Longer reaction times and lower yields in some steps.
iv) In prior art procedure intermediates are not isolated as solids in most of the steps and may lead to carryover of impurities to proceeding steps.

Based on the aforementioned drawbacks, prior art processes find to be unsuitable for preparation of fesoterodine at lab scale and commercial scale operations.

Hence, a need still remains for an improved and commercially viable process of preparing pure fesoterodine or a pharmaceutically acceptable salt thereof that will solve the aforesaid problems associated with process described in the prior art and will be suitable for large-scale preparation, in lesser reaction time, in terms of simplicity, purity and yield of the product.

### SUMMARY OF THE INVENTION

In one aspect, the present invention provides a convenient, commercially viable and environment friendly process for the preparation of Fesoterodine or a pharmaceutically acceptable salt thereof. Moreover, the reagents used for present invention are non-hazardous and easy to handle at commercial scale and also involves less reaction time. The process avoids tedious and cumbersome procedures of and convenient to operate on a commercial scale.

In another aspect, provided herein is an efficient, convenient, commercially viable and environment friendly resolution process for the preparation of enantiomerically pure fesoterodine intermediate, (R)—N,N-diisopropyl-3-(2-benzyloxy-5-halophenyl)-3-phenylpropylamine of formula VII.

In another aspect, provided herein is an efficient, convenient, commercially viable and environment friendly resolution process for the preparation of enantiomerically pure fesoterodine intermediate, (R)—N,N-diisopropyl-3-(2-benzyloxy-5-bromophenyl)-3-phenylpropylamine of formula VII(i).

In another aspect, the present invention provides (R)—N,N-diisopropyl-3-(2-benzyloxy-5-halophenyl)-3-phenylpropylamine having enantiomeric purity greater than about 98%, specifically greater than about 99.9%, more specifically greater than about 99.95%, and most specifically greater than about 99.98% measured by HPLC.

In still another aspect, the present invention also encompasses the use of enantiomerically pure (R)—N,N-diisopropyl-3-(2-benzyloxy-5-halophenyl)-3-phenylpropylamine obtained by the process of the present invention for preparing fesoterodine.

### DETAILED DESCRIPTION OF THE INVENTION

According to one aspect of the present invention, there is provided a process for preparing fesoterodine of formula I:

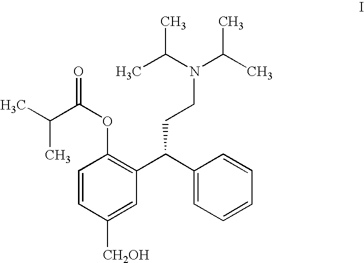

or a pharmaceutically acceptable salt thereof; which comprises:
a) reacting 4-phenylchroman compound of formula II:

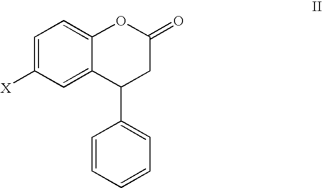

wherein ‘X’ represents a halogen atom, selected from the group consisting of F, Cl, Br and I;
with benzyl chloride in the presence of sodium iodide and a suitable inorganic base to give 3-phenylpropionate compound of formula III:

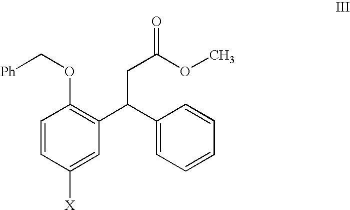

wherein ‘X’ is as defined for formula II;
b) reducing the compound of formula III obtained in step-(a) with a reducing agent in the presence of a Lewis acid to give hydroxy compound of formula IV:

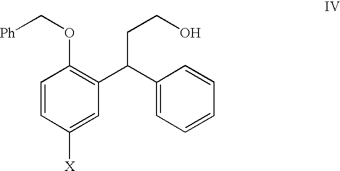

wherein ‘X’ is as defined for formula II;
c) reacting the compound of formula IV with a C₁-C₆-alkyl- or aryl-sulfonyl halide in the presence of an aliphatic organic base to give the protected compound of formula V:

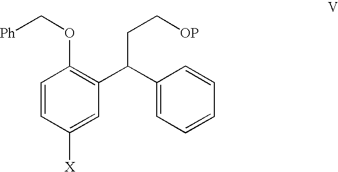

wherein ‘P’ represents a C₁-C₆-alkyl- or aryl-sulfonyl protecting group, and ‘X’ is as defined for formula II;
d) aminating the compound of formula V with diisopropylamine in a suitable organic solvent at a temperature ranging from 70° C.-140° C. in an autoclave or closed condition to give diisopropylamine compound of formula VI:

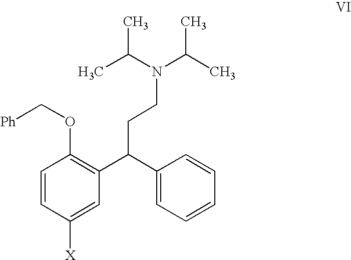

wherein ‘X’ is as defined for formula II;
e) resolving the compound of formula VI obtained in step-(d) with a suitable optically active acid to give (R)-enantiomer of formula VII:

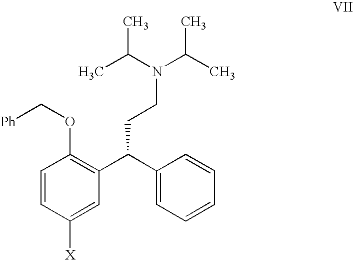

wherein ‘X’ is as defined for formula II;
f) reacting the (R)-enantiomer of formula VII with ethyl halide and magnesium in the presence of solid carbon dioxide to give (R)-4-benzyloxy-3-(3-diisopropylamino-1-phenylpropyl)-benzoic acid hydrochloride of formula VIII:

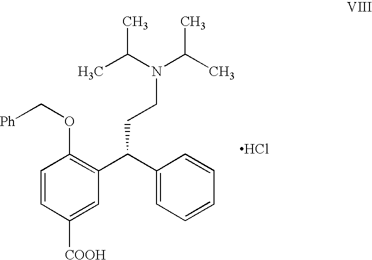

g) esterifying the compound obtained in step-(f) with a C₁-C₆-alcohol in the presence of acid chloride to obtain an ester compound of formula IX:

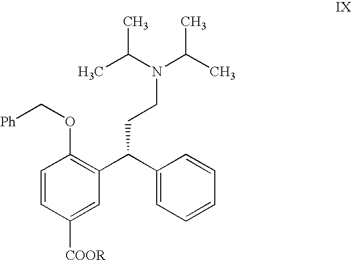

wherein ‘R’ represents C₁-C₆-alkyl-group such as methyl, ethyl and isopropyl;
h) reducing the compound of formula IX with a reducing agent in the presence of a Lewis acid to give (R)[4-benzyloxy-3-(3-diisopropylamino-1-phenylpropyl)-phenyl]-methanol of formula X:

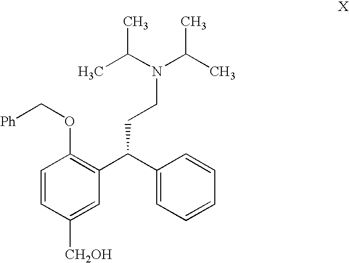

i) removing the benzyl protecting group of formula X to give (R)-2-(3-diisopropylamino-1-phenylpropyl)-4-hydroxymethylphenol of formula XI:

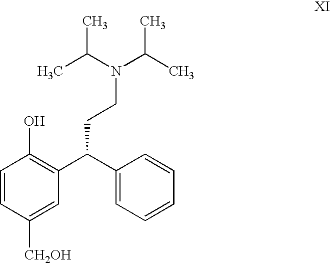

j) condensing the compound of formula XI with isobutyryl chloride in a suitable solvent, optionally in the presence of a suitable base, to produce substantially pure fesoterodine of formula I and optionally converting the fesoterodine formed in to a pharmaceutically acceptable acid addition salt of fesoterodine.

Preferably the halogen atom ‘X’ is Cl or Br, and more preferable halogen is Br.

6-Bromo-4-phenylchroman-2-one used as starting material in step-(a) may be obtained by processes described in the prior art, for example by the process described in the U.S. Pat. No. 5,559,269.

The term “substantially pure fesoterodine or a pharmaceutically acceptable salt thereof” refers to the fesoterodine or a pharmaceutically acceptable salt thereof having purity greater than about 99%, specifically greater than about 99.5%, and more specifically greater than about 99.9% (measured by HPLC).

The preferable inorganic bases used in step-(a) are hydroxides, carbonates, bicarbonates, alkoxides and oxides of alkali or alkaline earth metals. The preferred alkali metal compounds are those of lithium, sodium and potassium, more preferred being those of sodium and potassium. The preferred alkaline earth metal compounds are those of calcium and magnesium, more preferred being those of magnesium. Some examples of bases are sodium hydroxide, potassium hydroxide, magnesium hydroxide, magnesium oxide, sodium carbonate, potassium carbonate, sodium bicarbonate, potassium bicarbonate, sodium tert-butoxide and potassium tert-butoxide. The more preferred bases are sodium carbonate and potassium carbonate, and most preferred base is potassium carbonate.

In a preferred embodiment, the ester compound of formula III formed in step-(a) is isolated as solid from a suitable organic solvent by conventional method.

The organic solvent used to isolate the ester compound of formula III is an aliphatic or aromatic hydrocarbon solvent such as heptane, pentane, hexane, toluene, xylene, cyclohexane, petroleum ether and a mixture thereof. Preferable organic solvent is hexane.

The reducing agent used in step-(b) includes a metal hydride, with the proviso that the metal hydride does not include lithium aluminium hydride, such as sodium borohydride and sodium cyanoborohydride. Preferable Lewis acids used in step-(b) are aluminium chloride, calcium chloride, boron triflouride and zinc chloride, and more preferable Lewis acid is aluminium chloride.

Preferably the reduction reaction in step-(b) is carried out in an organic solvent. Preferable organic solvents are monoglyme, diglyme and aprotic solvents like tetrahydrofuran, ethers and a mixture thereof. More preferable organic solvent is monoglyme.

Preferably, the Lewis acid used in this step is about 0.2 to 2.0 equivalents with respect to sodium borohydride and addition of lewis acid carried out in two or more than two portions.

The reaction in step-(b) is carried out at a temperature between −20° C. and 50° C., preferably at a temperature between 0° C. and 40° C., and more preferably carried out at about 0-25° C.

Preferably, the reaction in step-(c) is carried out in a chlorinated solvent such as methylene dichloride at a temperature between 0° C. and 40° C., preferably at about 20-30° C., in the presence of an aliphatic organic base.

Preferable C₁-C₆-alkylsulfonyl halide used in step-(c) is methanesulfonyl halide. The term “aryl” used in step-(c) denotes a substituted or unsubstituted aromatic hydrocarbon group such as phenyl, naphthyl, anthryl, etc. Preferred aryl group according to the present invention is phenyl.

Preferable arylsulfonyl halides are C₁-C₆-alkyl-, C₁-C₆-alkoxy-, halogen or nitro substituted arylsulfonyl halides; more preferable substituted arylsulfonyl halides are toluene sulfonyl halide and p-nitrobenzene sulfonyl halide; and most preferred being p-toluenesulfonyl halide. Preferable halides are chloride, bromide or iodide, and more preferable halide is chloride.

Preferable aliphatic organic bases are triethyl amine, diisopropyl amine, dimethyl amine, monomethyl amine (gas or aqueous solution) and diisopropyl ethyl amine, and more preferable aliphatic organic base is triethylamine.

The organic solvent used in step-(d) is selected from the group consisting of nitriles such as acetonitrile, propionitrile and the like, and more preferable organic solvent is acetonitrile.

Preferably, the amination in step-(d) is carried out with diisopropylamine using acetonitrile as solvent in an autoclave to give formula VI. The reaction is carried out at a temperature ranging from 70° C.-140° C. in an autoclave or closed condition. The preferred temperature range is 90-100° C. in autoclave.

In another preferred embodiment, the compound of formula VI formed in step-(d) is isolated as solid from an organic solvent by conventional means. The organic solvent used for isolation is an alcoholic solvent such as methanol, ethanol, isopropyl alcohol, isoamyl alcohol and butanol, and more preferable alcoholic solvent is isopropyl alcohol.

The resolution in step-(e) is carried out by the methods known in the art. Preferably the resolution is carried out by the resolution method disclosed hereinafter.

Suitable optically active acids used in step-(e) include, but are not limited to, optically active tartaric acid derivatives such as di-aroyl-tartaric acid selected from the group comprising (−)-di-p-toluoyl-L-tartaric acid, (+)-di-p-toluoyl-D-tartaric acid, (−)-dibenzoyl-L-tartaric acid, (+)-dibenzoyl-D-tartaric acid, and hydrates thereof. More preferable optically active acid is (−)-di-p-toluoyl-L-tartaric acid.

The resolution in step-(e) is carried out in an appropriate solvent or a mixture of appropriate solvents. Appropriate solvents include water, acetone, acetonitrile, methanol, ethanol, isopropyl alcohol, tert-butanol, dichloromethane, chloroform, carbon tetrachloride, dimethylformamide, dimethylsulphoxide, ethyl acetate, toluene, xylene, pentane, hexane, heptane, ethyl ether, isopropyl ether, tetrahydrofuran, 1,4-dioxane, ethyleneglycol, 1,2-dimethoxyethane, and mixtures thereof, and in general, any solvent susceptible to being used in a chemical process. Specific solvents are methanol, ethanol, isopropyl alcohol, ethyl acetate, water and mixtures thereof, and more specifically water, isopropyl alcohol and a mixture thereof.

Preferably the ethyl halide used in step-(f) is ethyl chloride, ethyl bromide or ethyl iodide, and most preferable ethyl halide is ethyl bromide.

In another preferred embodiment, the compound of formula VIII formed in step-(f) is isolated as solid from an alcoholic solvent by conventional means. Preferable alcoholic solvents are methanol, ethanol, isopropyl alcohol, isoamyl alcohol and butanol, and more preferable alcoholic solvents are methanol and isopropyl alcohol.

In a still another preferred embodiment, the compound of formula VIII is isolated as free base directly from reaction mixture in step-(f) using an alcoholic solvent selected from methanol and isopropyl alcohol.

The esterification reaction in step-(g) is carried out at a temperature ranging from 0-70° C., and preferably carried out at about 55-65° C.

Preferable C₁-C₆-alcohol used in step-(g) is methanol, ethanol, isopropyl alcohol or butanol, and more preferable C₁-C₆-alcohol is methanol.

Preferably, about 0.80 to 3.0 moles of acid chloride per mole of the compound of formula VIII and more preferably about 1.0 to 2.8 moles of the acid chloride per mole of the compound of formula VIII is used.

Preferable acid chloride used in step-(g) is thionyl chloride or sulfonyl chloride, and more preferable acid chloride is thionyl chloride.

The reducing agent used in step-(h) includes a metal hydride, with the proviso that the metal hydride does not include lithium aluminium hydride, such as sodium borohydride and sodium cyanoborohydride. Preferable Lewis acids used in step-(h) are aluminium chloride, calcium chloride, boron triflouride and zinc chloride, and more preferable Lewis acid is aluminium chloride.

Preferably, the reduction reaction in step-(h) is carried out in an organic solvent. Preferable organic solvents are monoglyme, diglyme, aprotic solvents like tetrahydrofuran, ethers and a mixture thereof. Most preferable organic solvent is monoglyme.

Preferably, the Lewis acid used in this step is about 0.2 to 2.0 equivalents with respect to sodium borohydride and addition of Lewis acid is carried out in two or more than two portions.

The reaction in step-(h) is carried out at a temperature between −20° C. and 50° C., preferably at a temperature between 0° C. and 40° C., and more preferably carried out at about 0-15° C.

The removal of benzyl protecting group can be achieved by hydrogenation, the resulting material is isolated as solid by using a solvent selected from an ester solvent such as ethyl acetate and an ether solvent such as isopropyl ether in pure form and converted the resulting material to fesoterodine.

The condensation reaction in step-(j) can be carried out by the methods known in the art. The reaction is preferably carried out at a temperature of below about 50° C., more preferably at a temperature of about −20° C. to about 30° C. for at least 20 minutes, and still more preferably at a temperature of about −15° C. to about 15° C. from about 30 minutes to about 4 hours. Preferable solvents used in step-(j) include, but are not limited to, hydrocarbons, chlorinated hydrocarbons, nitriles, esters, ethers, and mixtures thereof, and most preferably methylene chloride.

The base used in step-(j) can be an organic or inorganic base. Preferable base is an organic base. Specific organic bases are organic amine bases of formula NR₁R₂R₃ wherein R₁, R₂ and R₃ are each independently hydrogen, C₁₋₆ straight or branched chain alkyl, aryl alkyl, C₃₋₁₀ single or fused ring optionally substituted, alkylcycloalkyls or independently R₁, R₂ and R₃ combine with each other to form C₃₋₇ membered cycloalkyl ring or heterocyclic system containing one or more heteroatom. Most preferable organic base is triethyl amine.

Exemplary inorganic bases include, but are not limited to, hydroxides, carbonates, alkoxides and bicarbonates of alkali or alkaline earth metals. Specific alkali metals are lithium, sodium and potassium, and more specifically sodium and potassium. Specific alkaline earth metals are calcium and magnesium, and more specifically magnesium. Specific inorganic bases are sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide, and more specifically sodium hydroxide, potassium hydroxide, sodium carbonate and potassium carbonate.

The reaction mass containing the pure fesoterodine of formula I obtained in step-(j) may be subjected to usual work up such as washings, extractions etc., followed by isolation from a suitable organic solvent by methods usually known in the art such as cooling, partial removal of the solvent from the solution, addition of precipitating solvent, or a combination thereof. Preferable organic solvents used for isolation include, but are not limited to, hydrocarbons, chlorinated hydrocarbons, nitriles, esters, ethers, and mixtures thereof, and most preferably methylene chloride.

Pharmaceutically acceptable salts of fesoterodine can be prepared in high purity by using the substantially pure fesoterodine free base obtained by the methods disclosed herein, by known methods.

Preferable pharmaceutically acceptable salts of fesoterodine include hydrochloride, hydrobromide, sulfate, fumarate and tartarate, and more preferably fumarate.

The total purity of the fesoterodine or a pharmaceutically acceptable salt thereof obtained by the process disclosed herein is of greater than about 99%, specifically greater than about 99.5%, and more specifically greater than about 99.9% as measured by HPLC.

Provided also herein is an improved resolution process for the preparation of (R)—N,N-diisopropyl-3-(2-benzyloxy-5-halophenyl)-3-phenylpropylamine compound of formula VII:

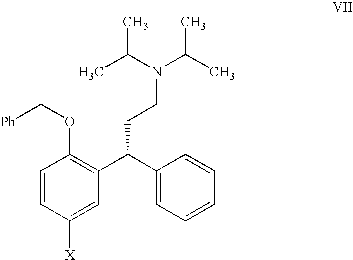

wherein ‘X’ represents a halogen atom selected from the group consisting of F, Cl, Br and I; or a salt thereof, which comprises:
a) reacting racemic (±)-N,N-diisopropyl-3-(2-benzyloxy-5-halophenyl)-3-phenyl propylamine compound of formula VI:

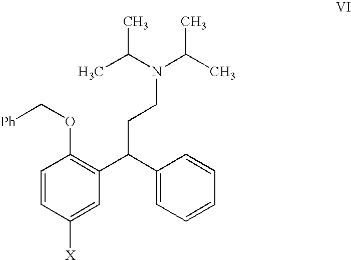

wherein ‘X’ is as defined for formula VII;
with a suitable optically active di-aroyl-tartaric acid in a suitable solvent, optionally in the presence of a suitable acid, to produce a diastereomeric excess of di-aroyl-tartaric acid salt compound of formula XII:

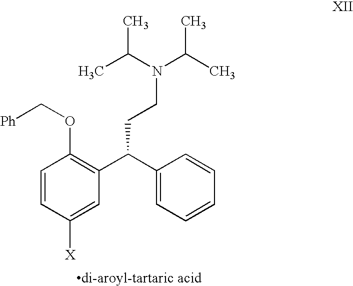

wherein ‘X’ is as defined for formula VII;
b) if required, separating the diastereomers of formula XII; and
c) neutralizing the product of step-(a) or separated diastereomers of step-(b) with a base in a suitable solvent to provide enantiomerically pure compound of formula VII.

The term “enantiomerically pure compound of formula VII” refers to the compound of formula VII having enantiomeric purity greater than about 98%, specifically greater than about 99.9%, more specifically greater than about 99.95%, and most specifically greater than about 99.98% measured by HPLC.

Preferably the halogen atom ‘X’ is Cl or Br, and more preferable halogen is Br.

The optically active di-aroyl-tartaric acid used in step-(a) is selected from the group comprising (−)-di-p-toluoyl-L-tartaric acid, (+)-di-p-toluoyl-D-tartaric acid, (−)-dibenzoyl-L-tartaric acid, (+)-dibenzoyl-D-tartaric acid, and hydrates thereof. More preferable optically active acid is (−)-di-p-toluoyl-L-tartaric acid.

The optically active di-aroyl-tartaric acid in step-(a) can be optionally used as a mixture with other acids (adjuvant acids) that can be organic or inorganic, such as hydrochloric acid, p-toluensulphonic acid, methanosulphonic acid or a mixture thereof, in molar proportions that vary between 0.5% and 50% (this molar percentage refers to the total of the mixture of the chiral acid and the adjuvant acid).

The reaction in step-(a) is carried out in an appropriate solvent or a mixture of appropriate solvents. Appropriate solvents include, but are not limited to, water, acetone, acetonitrile, methanol, ethanol, isopropyl alcohol, tert-butanol, dichloromethane, chloroform, carbon tetrachloride, dimethylformamide, dimethylsulphoxide, ethyl acetate, toluene, xylene, pentane, hexane, heptane, ethyl ether, isopropyl ether, tetrahydrofuran, 1,4-dioxane, ethyleneglycol, 1,2-dimethoxyethane, and mixtures thereof, and in general, any solvent susceptible to being used in a chemical process. Specific solvents are methanol, ethanol, isopropyl alcohol, ethyl acetate, water and mixtures thereof, and more specifically water, isopropyl alcohol and a mixture thereof.

The reaction in step-(a) is carried out at a temperature of −20° C. to the reflux temperature of the solvent used, specifically at a temperature of 0° C. to the reflux temperature of the solvent used, more specifically at a temperature of 20° C. to the reflux temperature of the solvent used, and most specifically at the reflux temperature of the solvent used.

The term “diastereomeric excess” refers to formation of a diastereomer having one configuration at chiral carbon of formula XII in excess over that having the opposite configuration. Preferably, one diastereomer is formed in above about 60% of the mixture of diastereomers over the other, and more preferably above about 80% of the mixture of diastereomers.

The compounds of formula XII formed may be used directly in the next step or the compounds of formula XII may be isolated from the reaction medium and then used in the next step.

The separation of diastereomers in step-(b) may be required to obtain stereomers with desired optical purity. It is well known that diastereomers differ in their properties such as solubility and then can be separated based on the differences in their properties. The separation of the diastereomers can be performed using the methods known to the person skilled in the art. These methods include chromatographic techniques and fractional crystallization, preferable method being fractional crystallization.

Preferably, a solution of the diastereomeric mixture is subjected to fractional crystallization. The solution of the diastereomeric mixture may be a solution of the reaction mixture obtained as above or a solution prepared by dissolving the isolated diastereomeric mixture in a solvent. Preferable solvents used for the separation include, but are not limited to, water; alcohols such as methanol, ethanol, isopropyl alcohol, propanol, tert-butyl alcohol, n-butanol; ketones such as acetone, methyl ethyl ketone, methyl isobutyl ketone, diethyl ketone; esters such as ethyl acetate, methyl acetate, isopropyl acetate, tert-butyl methyl acetate and ethyl formate; acetonitrile; tetrahydrofuran; dimethylformamide; dimethylsulfoxide; dioxane; diethyl carbonate; and mixtures thereof. Preferable solvents are water, methanol, ethanol, isopropyl alcohol, and mixtures thereof. More preferable solvents are water, isopropyl alcohol, and mixtures thereof.

Fractional crystallization of preferentially one diastereomer from the solution of mixture of diastereomers can be performed by conventional methods such as cooling, partial removal of solvents, using anti-solvent, seeding or a combination thereof.

Fractional crystallization can be repeated until the desired chiral purity is obtained. But, usually one or two crystallizations may be sufficient.

The base used in step-(c) can be an organic or inorganic base. Specific organic bases are triethyl amine, dimethyl amine and tert-butyl amine. Preferable base is an inorganic base. Exemplary inorganic bases include, but are not limited to, hydroxides, carbonates and bicarbonates of alkali or alkaline earth metals. Specific alkali metals are lithium, sodium and potassium, and more specifically sodium and potassium. Specific alkaline earth metals are calcium and magnesium, and more specifically magnesium.

Specific inorganic bases are sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide, and more specifically sodium hydroxide, potassium hydroxide, sodium carbonate and potassium carbonate.

Exemplary solvents for step-(c) include, but are not limited to, water, alcohols, ketones, cyclic ethers, aliphatic ethers, hydrocarbons, chlorinated hydrocarbons, nitriles, esters and the like, and mixtures thereof. Specific solvents are water, hydrocarbons, alcohols, chlorinated hydrocarbons, and mixtures thereof.

Exemplary alcohol solvents include, but are not limited to, C₁ to C₈ straight or branched chain alcohol solvents such as methanol, ethanol, propanol, butanol, amyl alcohol, hexanol, and mixtures thereof. Specific alcohol solvents are methanol, ethanol, isopropyl alcohol, and mixtures thereof, and most specific alcohol solvent is isopropyl alcohol. Exemplary ketone solvents include, but are not limited to, acetone, methyl isobutyl ketone, and the like, and mixtures thereof. Exemplary cyclic ether solvents include, but are not limited to, tetrahydrofuran, dioxane, and the like, and mixtures thereof. Exemplary nitrile solvents include, but are not limited to, acetonitrile and the like, and mixtures thereof. Exemplary ester solvents include, but are not limited to, ethyl acetate, isopropyl acetate, and the like and mixtures thereof. Exemplary hydrocarbon solvents include, but are not limited to, n-pentane, n-hexane and n-heptane and isomers or mixtures thereof, cyclohexane, toluene and xylene. Specific hydrocarbon solvent is toluene. Exemplary chlorinated hydrocarbon solvents include, but are not limited to, methylene chloride, ethyl dichloride, chloroform and carbon tetrachloride or mixtures thereof. Specific chlorinated hydrocarbon solvent is methylene chloride.

Preferable solvent for step-(c) is selected from the group consisting of water, methylene chloride, n-hexane, n-heptane, cyclohexane, toluene, xylene, and mixtures thereof.

The reaction mass containing the enantiomerically pure compound of formula VII obtained in step-(c) may be subjected to usual work up such as washings, extractions etc., followed by isolation from a suitable organic solvent by methods usually known in the art such as cooling, partial removal of the solvent from the solution, addition of precipitating solvent, or a combination thereof.

In an embodiment, the resolution procedure of the present invention can be used to resolve mixtures that comprise both enantiomers of the compound of formula VI in any proportion. Therefore, this procedure is applicable both to performing the optical resolution of a racemic mixture of the compound of formula VI (that is to say, that in which the two enantiomers are present in a 1:1 ratio) and for the optical resolution of non-racemic mixtures of the compound of formula VI (in which one of the enantiomers is present in greater proportion), obtained by any physical or chemical method.

In particular, most preferred compound of formula VII prepared by the process described herein is the (R)—N,N-diisopropyl-3-(2-benzyloxy-5-bromophenyl)-3-phenylpropylamine of formula VII(i) (formula VII, wherein X is Br). The compound of formula VII(i):

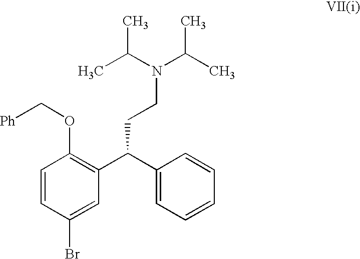

is useful intermediate for the preparation of fesoterodine of formula I.

The enantiomeric purity of the compound of formula VII, preferably (R)—N,N-diisopropyl-3-(2-benzyloxy-5-bromophenyl)-3-phenylpropylamine of formula VII(i), obtained by the process disclosed herein is of greater than about 98%, specifically greater than about 99.9%, more specifically greater than about 99.95%, and most specifically greater than about 99.98% measured by HPLC.

Fesoterodine and pharmaceutically acceptable salts of Fesoterodine can be prepared in high purity by using the enantiomerically pure (R)—N,N-diisopropyl-3-(2-benzyloxy-5-halophenyl)-3-phenylpropylamine compound of formula VII or its acid addition salts thereof obtained by the methods disclosed herein, by known methods.

Aptly the processes of this invention are adapted to the preparation of fesoterodine or a pharmaceutically acceptable salt thereof in high enantiomeric and chemical purity.

The following examples are provided to enable one skilled in the art to practice the invention and are merely illustrate the process of this invention. However, it is not intended in any way to limit the scope of the present invention.

### EXAMPLES

### Example 1

### Step-1: Preparation of 6-Bromo-4-phenylchroman-2-one

### Method-A:

Cinnamic acid (100 g, 676 mmol), 4-bromophenol (123 g, 730 mmol) and sulfuric acid (13 ml) were taken into a 1 L 4-neck round bottom flask. The contents were slowly heated to 120-125° C. and stirred for 3 to 4 hours at 120-125° C. The reaction mixture was cooled to 80° C. followed by the addition of toluene (300 ml) and water (200 ml) and then stirred for 15 minutes. The toluene layer was separated and washed with water (2×100 ml). The resulting toluene layer was distilled completely under vacuum. Potassium carbonate solution (47% w/v-100 ml) was added to the residue at 25-30° C., the contents were stirred for 15 minutes, filtered the solid and washed with water (2×100 ml). The wet material was leached with 100 ml of isopropyl alcohol and then filtered. The resulting solid was washed with 50 ml of isopropyl alcohol and then dried the material at 70-75° C. to give 72 g of 6-bromo-4-phenylchroman-2-one (Melting poing: 117° C.; HPLC Purity: 98.5%).

### Method-B:

Cinnamic acid (100 g, 676 mmol), 4-bromophenol (135 g, 801 mmol) and sulfuric acid (15 ml) were taken into a 2 L 4-neck round bottom flask. The contents were slowly heated to 120-125° C. and stirred for 3 to 4 hours at 120-125° C. The reaction mixture was cooled to 80° C. followed by the addition of toluene (1000 ml) and water (300 ml) and then stirred for 15 minutes. The toluene layer was separated and washed with aqueous sodium chloride solution (3×200 ml). The toluene layer was distilled completely under vacuum to give residue. Isopropyl alcohol (300 ml) was added to the residue and then stirred at 55-60° C. for 30 minutes. The resulting mass was cooled to 0-5° C. and then stirred for 1 hour at 0-5° C. The resulting solid was filtered, washed with isopropyl alcohol (200 ml) and then dried the product at 55-60° C. to give 150 g of 6-bromo-4-phenylchroman-2-one (Melting poing: 117° C.; HPLC Purity: 99.66%).

### Step-2: Preparation of Methyl(±)-3-(2-benzyloxy-5-bromophenyl)-3-phenyl Propionate

6-Bromo-4-phenylchroman-2-one (100 g, 330 mmole), potassium carbonate (55.28 g, 400 mmole), sodium iodide (24.72 g, 165 mmole), benzyl chloride (47.6 g, 376 mmole), acetone (412 ml) and methanol (412 ml) were taken into a reaction flask. The contents were heated to reflux and stirred for 5-6 hours. The solvents were removed completely under vacuum and followed by the addition of dichloromethane (300 ml) and washed the organic layer with water. The solvent was distilled off completely under vacuum, n-hexane (220 ml) was added to the oily mass and then stirred for 2-3 hours at 25-30° C. The material was filtered, washed with 50 ml of n-hexane and then dried the solid to give 124 g of methyl(±)-3-(2-benzyloxy-5-bromophenyl)-3-phenylpropionate (Yield: 88.5%; HPLC Purity: 99.53%).

### Step-3: Preparation of (±)-3-(2-Benzyloxy-5-bromophenyl)-3-phenylpropan-1-ol

Methyl(±)-3-(2-benzyloxy-5-bromophenyl)-3-phenylpropionate (100 g, 235 mmole), sodium borohydride (10.66 g, 282 mmole) and monoglyme (300 ml) were taken into a reaction flask. The reaction mixture was stirred for 10 minutes and cooled to 10° C. This was followed by the addition of aluminium chloride (15.633 g, 118 mmole) portion wise at below 10° C. over a period of 2 hours and stirred for 1 hour at 10° C. Diluted hydrochloric acid was added drop wise to the reaction mass at below 5° C. followed by the addition of dichloromethane (900 ml) and then stirred for 10 minutes. The layers were separated and the organic layer was washed with water followed by distillation under vacuum to get oily mass. Petroleum ether (100 ml) was added to oily mass and stirred for 1 hour at 25-30° C., filtered and washed with 50 ml of petroleum ether. The material was dried at below 60° C. to give 88 g of (±)-3-(2-benzyloxy-5-bromophenyl)-3-phenylpropan-1-ol (Yield: 93.6%; HPLC Purity: 99.23%).

### Step-4: Preparation of (±)-Toluene-4-sulphonic acid 3-(2-benzyloxy-5-bromophenyl)-3-phenylpropyl ester

(±)-3-(2-Benzyloxy-5-bromophenyl)-3-phenylpropan-1-ol (100 g, 252 mmole), dichloromethane (500 ml), triethylamine (53 ml, 378 mmole) and p-toluenesulphonyl chloride (50.4 g, 265 mmole) were taken into a round bottom flask at 25-30° C. and stirred for 12 hours at 25-30° C. Water (250 ml) was added to the reaction mass and the pH of aqueous layer was adjusted to 3-4 with hydrochloric acid. The layers were separated and the organic layer was dried with sodium sulphate. The dichloromethane layer was concentrated under vacuum to obtain 138 g of (±)-toluene-4-sulphonic acid 3-(2-benzyloxy-5-bromophenyl)-3-phenylpropyl ester as oily residue (HPLC Purity: 95%).

### Step-5: Preparation of (±)-N,N-Diisopropyl-3-(2-benzyloxy-5-bromophenyl)-3-phenylpropylamine

### Method-A:

(±)-Toluene-4-sulphonic acid 3-(2-benzyloxy-5-bromophenyl)-3-phenylpropyl ester (100 g, 181.4 mmole) was dissolved in a mixture of acetonitrile (250 ml) and diisopropylamine (180 g, 1777 mmole), and the mixture was heated at 95-100° C. under closed conditions in an autoclave for 30 hours. The reaction mixture was cooled to 0° C. and stirred for 1 hour followed by filtration and then washed with acetonitrile (100 ml). Acetonitrile was distilled off completely under vacuum to get oily mass. Water (300 ml) was added to oily mass and adjusted the pH with hydrochloric acid to 1-2 to get two layers. The oily layer was separated and dissolved into water (300 ml) and then washed with ether (200 ml). The aqueous layer was separated and the pH was adjusted with ammonia solution to 9-10 and then extracted with dichloromethane (300 ml). The dichloromethane was distilled off completely under vacuum and followed by the addition of isopropyl alcohol to the residue. The reaction mass was stirred for 1 hour at 25-30° C., filtered the solid, washed with isopropyl alcohol and then dried at 60° C. to give 55 g of (±)-N,N-diisopropyl-3-(2-benzyloxy-5-bromophenyl)-3-phenylpropylamine (HPLC Purity: 93.8%).

### Method-B:

(±)-Toluene-4-sulphonic acid 3-(2-benzyloxy-5-bromophenyl)-3-phenylpropyl ester (100 g, 181.4 mmole) was dissolved in a mixture of acetonitrile (250 ml) and diisopropylamine (180 g, 1777 mmole), and the mixture was heated to 95-100° C. under closed conditions in an autoclave for 30 hours. The reaction mixture was cooled to 0° C. and stirred for 1 hour. The resulting mass was filtered and washed with acetonitrile (100 ml). The acetonitrile was distilled off completely under vacuum to get oilymass. The oily mass was stirred for 10 minutes and followed by the addition of methylene dichloride (250 ml) and water (200 ml). The pH of the aqueous layer was adjusted to 1-2 with hydrochloric acid. The layers were separated and water (200 ml) was added to methylene dichloride layer. The pH of the aqueous layer was adjusted to 9 with ammonia solution and followed by separation of the layers. The organic layer was distilled under vacuum at below 50° C. to give 65 g of (±)-N,N-diisopropyl-3-(2-benzyloxy-5-bromophenyl)-3-phenylpropylamine as oily mass (HPLC Purity: 94%).

### Step-6: Resolution of (±)-N,N-Diisopropyl-3-(2-benzyloxy-5-bromophenyl)-3-phenylpropylamine (crude salt and its purification)

### Method-A:

(±)-N,N-Diisopropyl-3-(2-benzyloxy-5-bromophenyl)-3-phenylpropylamine (100 g, 208 mmole) and isopropyl alcohol (1500 ml) were taken into a round bottom flask. Di-p-toluoyl-L-tartaric acid (80 g, 207 mmole) was added to the above mass and heated to reflux. The reaction mass was stirred for 1 hour at 86° C. and then slowly cooled to 25-30° C. After being stirred at 25-30° C. for 14 hours, formed salts were filtered, washed with isopropyl alcohol and then dried to give 85 g of levorotatory salt [Melting Range: about 120° C.; S.O.R: (−68°, C=1, Methanol)].

### Method-B:

(±)-N,N-Diisopropyl-3-(2-benzyloxy-5-bromophenyl)-3-phenylpropylamine (100 g) and isopropyl alcohol (800 ml) were taken into a round bottom flask. The contents were heated to 55-60° C. and followed by the addition of (−)-di-p-toluoyl-L-tartaric acid (73 g). The reaction mixture was heated to 80-85° C. and stirred for 1 hour at 80-85° C. The resulting mass was cooled to 40-45° C., filtered and washed with isopropyl alcohol (200 ml). The resulting wet material was added to a mixture of isopropyl alcohol (855 ml) and water (95 ml) and then heated to 80-85° C. and stirred for 1 hour. The resulting mass was cooled to 40-45° C. and the solid was filtered, washed with a mixture of isopropyl alcohol (200 ml) and water (20 ml), and then dried to give pure salt [S.O.R=(−69.2° C=1, Methanol)].

### Step-7: Preparation of (R)—N,N-Diisopropyl-3-(2-benzyloxy-5-bromophenyl)-3-phenylpropylamine

### Method-A:

The levorotatory salt (85 g, obtained in method-A of step-6) was dissolved in water and basified with 2N NaOH solution. The resulting solution was extracted with dichloromethane, dried with sodium sulphate and distilled under vacuum to give 40 g of (R)—N,N-diisopropyl-3-(2-benzyloxy-5-bromophenyl)-3-phenylpropylamine as colour less oil. [Yield: 83%); [a]²⁴=−14° (C=5, ethanol, as on basis)].

### Method-B:

The levorotatory salt (100 g, obtained in method-B of step-6) was dissolved in water (500 ml) and basified with sodium carbonate (31 g) to get pH 9-10. The solution was extracted with dichloromethane, washed with brine solution and dried with sodium sulphate followed by distillation under vacuum to give 36 g of (R)—N,N-diisopropyl-3-(2-benzyloxy-5-bromophenyl)-3-phenylpropylamine as colorless oil.

[Yield=75%); [a]²⁴=−16° (C=5, ethanol); HPLC Purity: 99.6%].

### Step-8: Preparation of (R)-4-Benzyloxy-3-(3-diisopropylamino-1-phenylpropyl)-benzoic acid hydrochloride

A mixture of Magnesium (26 g), ethyl bromide (0.6 ml), iodine (2 crystals) and tetrahydrofuran (200 ml) was heated at 55-60° C. for initiation of reaction. This was followed by drop wise addition of a solution of (R)—N,N-diisopropyl-3-(2-benzyloxy-5-bromophenyl)-3-phenylpropylamine (100 g, 208.3 mmole) and ethyl bromide (54 ml) in tetrahydrofuran (500 ml). The reaction mixture was refluxed for 1 hour and cooled to −65° C. This was followed by the addition of powdered dry Ice (100 g) at below −60° C. and stirred for 1 hour. Ammonium chloride solution (20%, 700 ml) was added to the reaction mixture at below 0° C. and stirred for 30 minutes. The layers were separated and the aqueous layer was washed with 100 ml of ether. The pH of aqueous layer was adjusted with diluted hydrochloric acid to 1-2 and extracted the product with dichloromethane (300 ml). The resulting organic layer was washed with water and distilled out completely under vacuum. Methanol was added to the residue, filtered the precipitated product and dried to give 65 g of (R)-4-Benzyloxy-3-(3-diisopropylamino-1-phenylpropyl)-benzoic acid hydrochloride (HPLC Purity: 96.73%).

### Step-9: Preparation of methyl(R)-4-benzyloxy-3-(3-diisopropylamino-1-phenylpropyl)-benzoate

Methanol (1000 ml) and (R)-4-Benzyloxy-3-(3-diisopropylamino-1-phenylpropyl)-benzoic acid hydrochloride (100 g, 225 mmole) were taken into a round bottom flask. The contents were cooled to 10° C. followed by drop wise addition of thionyl chloride (37 g). The reaction mixture was slowly heated and refluxed for 2-3 hours. Methanol was distilled off completely under vacuum and then 300 ml of dichloromethane was added to the oily mass. The dichloromethane layer was washed with saturated sodium bicarbonate solution (2×50 ml) followed by water washings. The dichloromethane solvent was distilled off completely under vacuum to give methyl(R)-4-benzyloxy-3-(3-diisopropylamino-1-phenylpropyl)-benzoate as oily mass. This was followed by the addition of a mixture of isopropyl alcohol (250 ml) and water (250 ml), and stirred for one hour at 55-60° C. The resulting mass was cooled to 25-30° C. and stirred at 25-30° C. The separated solid was filtered, washed with a mixture of isopropyl alcohol (50 ml) and water (50 ml) and then dried the material at 60° C. to give 75 g of methyl(R)-4-benzyloxy-3-(3-diisopropylamino-1-phenylpropyl)-benzoate (HPLC Purity: 99.52%).

### Step-10: Preparation of (R)-[4-benzyloxy-3-(3-diisopropylamino-1-phenylpropyl)-phenyl]-methanol

Methyl(R)-4-benzyloxy-3-(3-diisopropylamino-1-phenylpropyl)-benzoate (100 g, 218 mmole) in tetrahydrofuran (500 ml), sodium borohydride (10.66 g, 282 mmole) and monoglyme (300 ml) were taken into a reaction flask. The contents were stirred for 10 minutes and then cooled to 10° C. Aluminium chloride (15.633 g, 118 mmole) was added portion wise at below 10° C. over a period of 2 hours and stirred for 1 hour at 10° C. Dilute hydrochloric acid was added drop wise to the reaction mass at below 5° C. followed by addition of dichloromethane (900 ml) and stirred for 10 minutes. The layers were separated and the organic layer was washed with water followed by distillation under vacuum to give 94 g of (R)-[4-benzyloxy-3-(3-diisopropylamino-1-phenylpropyl)-phenyl]-methanol (HPLC Purity: 99.44%).

### Step-11: Preparation of (R)-2-(3-diisopropylamino-1-phenylpropyl)-4-hydroxy methylphenol

(R)-[4-Benzyloxy-3-(3-diisopropylamino-1-phenylpropyl)-phenyl]-methanol (100 g, 232 mmole) and methanol (1000 ml) were taken into a Parhydrogenator. Palladium carbon (5%, 20 g) was added and the mixture was hydrogenated with 2-3 kg pressure at 50-55° C. till the completion of reaction. The mixture was then filtered and the solvent was removed by vacuum at below 50° C. The resulting oil was dissolved in dichloromethane (100 ml) and the dichloromethane solution was washed with water, dried over sodium sulfate and evaporated to give 78 g of color less oil. This was crystallized in ethyl acetate and n-hexane to give (R)-2-(3-diisopropylamino-1-phenylpropyl)-4-hydroxy methylphenol as solid (Yield: 98%; HPLC Purity: 99.94%).

### Step-12: Preparation of (R)—N,N-Diisopropyl-3-(2-benzyloxy-5-bromophenyl)-3-phenylpropylamine (Fesoterodine)

(R)-2-(3-Diisopropylamino-1-phenylpropyl)-4-hydroxymethylphenol (100 g, 292 mmole) was added to dichloromethane (2000 ml) and cooled to 0° C. This was followed by the addition of a solution of isobutyryl chloride (31.1 g, 292 mmole) in dichloromethane (100 ml) at 0-5° C. over a period of 1 hour. The contents were stirred for 30 minutes followed by drop wise addition of a solution of triethyl amine (34.5 g, 297 mmole) in 50 ml of dichloromethane at 0-5° C. for 30 minutes. The resulting mass was stirred for 30 minutes, water (100 ml) was added, separated the layers and washed the dichloromethane layer with 5% sodium bicarbonate solution (100 ml). The dichloromethane layer was dried with sodium sulfate and then distilled off dichloromethane under vacuum to give 115 g of fesoterodine as oily mass (Yield: 95%).

### Step-13: Preparation of Fesoterodine Fumarate

A solution of Fesoterodine (42 g) in methyl ethyl ketone (90 ml) was stirred with fumaric acid (12 g) at 80° C. for 1 hour. This was followed by the slow addition of cyclohexane (30 ml) under stirring and further stirred for 1 hour at 80° C. The solution was cooled slowly to 25-30° C. and stirred for 6 hours at the same temperature. The solution was further cooled at 0-5° C. and stirred for overnight. The separated solid was filtered and washed with mixture of cyclohexane and Methyl ethyl ketone mixture to give fesoterodine fumarate (HPLC Purity: 99.88%).

### Example 2

### Step-1: Resolution of (±)-N,N-Diisopropyl-3-(2-benzyloxy-5-bromophenyl)-3-phenylpropylamine

### Method-A:

(±)-N,N-Diisopropyl-3-(2-benzyloxy-5-bromophenyl)-3-phenylpropylamine (100 g) was dissolved in isopropyl alcohol (1500 ml). This was followed by the addition of (−)-di-p-toluoyl-L-tartaric acid (80 g). The reaction mixture was further heated at 80° C. and refluxed for 1 hour. The reaction mixture was then stirred for 12 hours at 25-30° C. The resulting solid was filtered, washed with isopropyl alcohol and then dried to produce 85 g of (R)—N,N-Diisopropyl-3-(2-benzyloxy-5-bromophenyl)-3-phenylpropylamine di-p-toluoyl-L-tartrate salt [Melting Range: 120-125° C.; Specific optical rotation: (−60°, C=1, Methanol)].

### Purification Method-1:

The mixture of (R)—N,N-Diisopropyl-3-(2-benzyloxy-5-bromophenyl)-3-phenylpropylamine di-p-toluoyl-L-tartrate salt (85 g) and isopropyl alcohol (350 ml) was heated at 80° C. for 2 hours. The reaction mixture was cooled at 25-30° C. and stirred for 1 hour. The resulting solid was filtered and washed with isopropyl alcohol (170 ml). The resulting solid was further dried to give 76 g of pure salt [S.O.R: (−67.5°, C=1, Methanol)].

### Purification Method-2:

The mixture of (R)—N,N-Diisopropyl-3-(2-benzyloxy-5-bromophenyl)-3-phenylpropylamine di-p-toluoyl-L-tartrate salt (85 g), isopropyl alcohol (800 ml) and water (85 ml) was heated at 80° C. to get clear solution. The reaction mixture was stirred for 30 minutes at 78-82° C. The resulting mass was then cooled to 25-30° C. for 1 hour and stirred for 2 hours. The resulting solid was filtered and washed with 10% aqueous isopropyl alcohol (85 ml) and then dried to give 72 g of pure salt [S.O.R=(−69.2°, C=1, Methanol)].

### Method-B:

(±)-N,N-Diisopropyl-3-(2-benzyloxy-5-bromophenyl)-3-phenylpropylamine (100 g) and isopropyl alcohol (800 ml) were taken into a round bottom flask. The contents were heated to 55-60° C. and followed by the addition of (−)-di-p-toluoyl-L-tartaric acid (73 g). The reaction mixture was heated to 80-85° C. and stirred for 1 hour at 80-85° C. The resulting mass was cooled to 40-45° C., filtered and washed with isopropyl alcohol (200 ml). The resulting wet material was added to a mixture of isopropyl alcohol (855 ml) and water (95 ml) and then heated to 80-85° C. and stirred for 1 hour. The resulting mass was cooled to 40-45° C. and the solid was filtered, washed with a mixture of isopropyl alcohol (200 ml) and water (20 ml), and then dried to give pure salt [S.O.R=(−69.2°, C=1, Methanol)].

### Step-2: Preparation of (R)—N,N-Diisopropyl-3-(2-benzyloxy-5-bromophenyl)-3-phenylpropylamine

### Method-A:

Pure (R)—N,N-Diisopropyl-3-(2-benzyloxy-5-bromophenyl)-3-phenylpropylamine di-p-toluoyl-L-tartrate salt (76 g, obtained after purification in step-1) was dissolved in water and basified with 2N NaOH solution to get pH 9-10. The solution was extracted with dichloromethane, dried with sodium sulphate and then distilled under vacuum to give 36 g of (R)—N,N-Diisopropyl-3-(2-benzyloxy-5-bromophenyl)-3-phenylpropylamine as colorless oil. [Yield=75%; [a]²⁴=−16° (C=5, ethanol); HPLC Purity: 99.4%].

### Method-B:

Pure (R)—N,N-Diisopropyl-3-(2-benzyloxy-5-bromophenyl)-3-phenylpropylamine di-p-toluoyl-L-tartrate salt (100 g, obtained after purification in step-1) was dissolved in water (500 ml) and basified with sodium carbonate (31 g) to get pH 9-10. The solution was extracted with dichloromethane, washed with brine solution and dried with sodium sulphate followed by distillation under vacuum to give 36 g of (R)—N,N-diisopropyl-3-(2-benzyloxy-5-bromophenyl)-3-phenylpropylamine as colorless oil.

[Yield=75%); [a]²⁴=−16° (C=5, ethanol); HPLC Purity: 99.6%].

We claim:

1. A process for the preparation of fesoterodine of formula I: 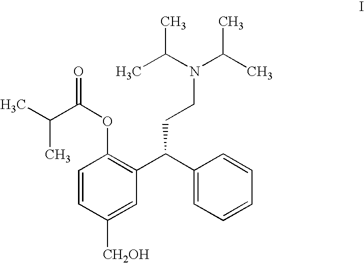
or a pharmaceutically acceptable salt thereof; which comprises:
a) reacting 4-phenylchroman compound of formula II: 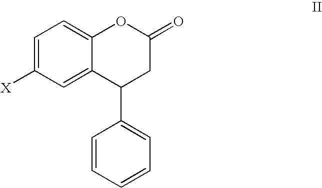
wherein ‘X’ represents a halogen atom, selected from the group consisting of F, Cl, Br and I; with benzyl chloride in the presence of sodium iodide and a suitable inorganic base to give 3-phenylpropionate compound of formula III: 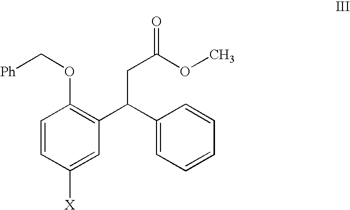
wherein ‘X’ is as defined for formula II;
b) reducing the compound of formula III obtained in step-(a) with a reducing agent in the presence of a Lewis acid to give hydroxy compound of formula IV: 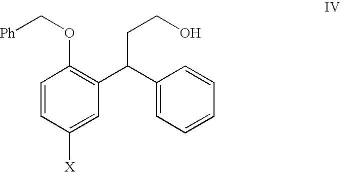
wherein ‘X’ is as defined for formula II;
c) reacting the compound of formula IV with a C₁-C₆-alkyl- or aryl-sulfonyl halide in the presence of an aliphatic organic base to give the protected compound of formula V: 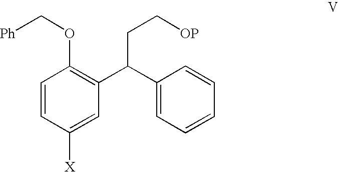
wherein ‘P’ represents a C₁-C₆-alkyl- or aryl-sulfonyl protecting group, and ‘X’ is as defined for formula II;
d) aminating the compound of formula V with diisopropylamine in a suitable organic solvent at a temperature ranging from 70° C.-140° C. in an autoclave or closed condition to give diisopropylamine compound of formula VI: 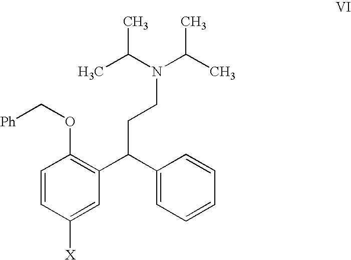
wherein ‘X’ is as defined for formula II;
e) resolving the compound of formula VI obtained in step-(d) with a suitable optically active acid to give (R)-enantiomer of formula VII: 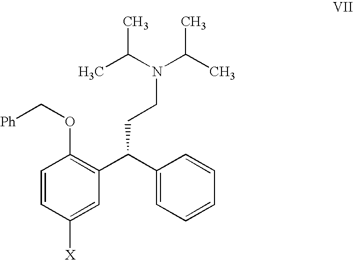
wherein ‘X’ is as defined for formula II;
f) reacting the (R)-enantiomer of formula VII with ethyl halide and magnesium in the presence of solid carbon dioxide to give (R)-4-benzyloxy-3-(3-diisopropylamino-1-phenylpropyl)-benzoic acid hydrochloride of formula VIII: 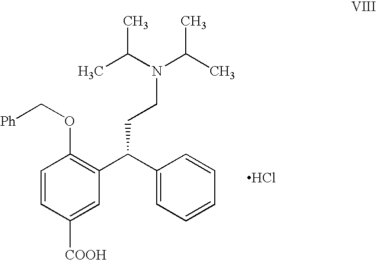
g) esterifying the compound obtained in step-(f) with a C₁-C₆-alcohol in the presence of acid chloride to obtain an ester compound of formula IX: 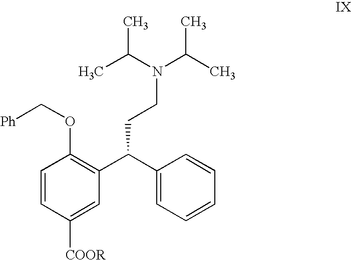
wherein ‘R’ represents C₁-C₆-alkyl-group such as methyl, ethyl and isopropyl;
h) reducing the compound of formula IX with a reducing agent in the presence of a Lewis acid to give (R)-[4-benzyloxy-3-(3-diisopropylamino-1-phenylpropyl)-phenyl]-methanol of formula X: 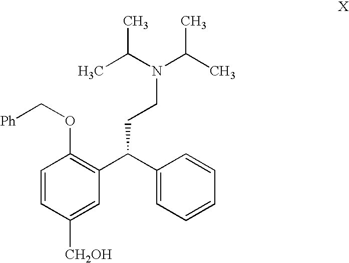
i) removing the benzyl protecting group of formula X to give (R)-2-(3-diisopropylamino-1-phenylpropyl)-4-hydroxymethylphenol of formula XI: 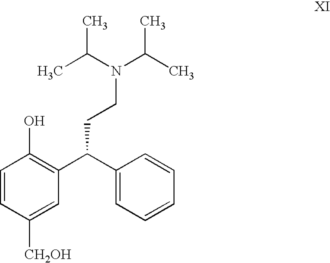
j) condensing the compound of formula XI with isobutyryl chloride in a suitable solvent, optionally in the presence of a suitable base, to produce substantially pure fesoterodine of formula I and optionally converting the fesoterodine formed in to a pharmaceutically acceptable acid addition salt of fesoterodine.

2. The process of claim 1, wherein the halogen atom ‘X’ is Br; wherein the reducing agent used in steps-(b) and (h) is a metal hydride, with the proviso that the metal hydride does not include lithium aluminium hydride, selected from the group comprising sodium borohydride and sodium cyanoborohydride; wherein the Lewis acid used in steps-(b) and (h) is selected from the group comprising aluminium chloride, calcium chloride, boron triflouride and zinc chloride; wherein the arylsulfonyl halide used in step-(c) is toluenesulfonyl chloride; wherein the aliphatic organic base used in step-(c) is selected from the group consisting of triethyl amine, diisopropyl amine, dimethyl amine, monomethyl amine (gas or aqueous solution) and diisopropyl ethyl amine; wherein the optically active acid used in step-(e) is a derivative of tartaric acid selected from the group comprising (−)-di-p-toluoyl-L-tartaric acid, (+)-di-p-toluoyl-D-tartaric acid, (−)-dibenzoyl-L-tartaric acid, (+)-dibenzoyl-D-tartaric acid, and hydrates thereof; and wherein the acid chloride used in step-(g) is selected from the group consisting of thionyl chloride and sulfonyl chloride.

3. (canceled)

4. The process of claim 2, wherein the metal hydride is sodium borohydride; wherein the Lewis acid is aluminium chloride; wherein the aliphatic organic base is triethyl amine; wherein the optically active acid is (−)-di-p-toluoyl-L-tartaric acid; and wherein the acid chloride is thionyl chloride.

5. (canceled)

6. (canceled)

7. The process of claim 1, wherein the Lewis acid used in steps-(b) and (h) is about 0.2 to 2.0 equivalents per one equivalent of sodium borohydride; wherein the acid chloride in step-(g) is used in a molar ratio of about 0.80 to 3.0 moles per one mole of the compound of formula VIII; and wherein the fesoterodine or a pharmaceutically acceptable salt thereof obtained has a total purity of greater than about 99% as measured by HPLC.

8. The process of claim 1, wherein the reaction in steps-(b) and (h) is carried out in an organic solvent selected from the group comprising monoglyme, diglyme, tetrahydrofuran, ethers, and mixtures thereof; wherein the reaction in step-(c) is carried out in a chlorinated solvent; wherein the organic solvent used in step-(d) is a nitrile solvent selected from the group consisting of acetonitrile and propionitrile; and wherein the resolution in step-(e) is carried out in a solvent selected from the group consisting of water, acetone, acetonitrile, methanol, ethanol, isopropyl alcohol, tert-butanol, dichloromethane, chloroform, carbon tetrachloride, dimethylformamide, dimethylsulphoxide, ethyl acetate, toluene, xylene, pentane, hexane, heptane, ethyl ether, isopropyl ether, tetrahydrofuran, 1,4-dioxane, ethyleneglycol, 1,2-dimethoxyethane, and mixtures thereof.

9. The process of claim 8, wherein the organic solvent used in steps-(b) and (h) is monoglyme; and wherein the solvent used in step-(e) is selected from the group consisting of methanol, ethanol, isopropyl alcohol, ethyl acetate, water and mixtures thereof.

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. A resolution process for the preparation of (R)—N,N-diisopropyl-3-(2-benzyloxy-5-halophenyl)-3-phenylpropylamine compound of formula VII: 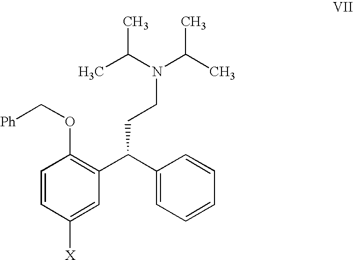
wherein ‘X’ represents a halogen atom selected from the group consisting of F, Cl, Br and I; or a salt thereof, which comprises:
a) reacting racemic (±)-N,N-diisopropyl-3-(2-benzyloxy-5-halophenyl)-3-phenyl propylamine compound of formula VI: 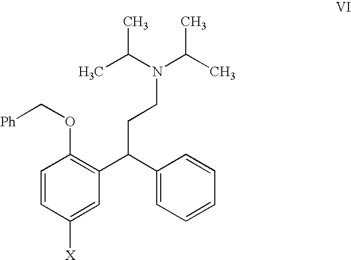
wherein ‘X’ is as defined for formula VII;
with a suitable optically active di-aroyl-tartaric acid in a suitable solvent, optionally in the presence of a suitable acid, to produce a diastereomeric excess of di-aroyl-tartaric acid salt compound of formula XII: 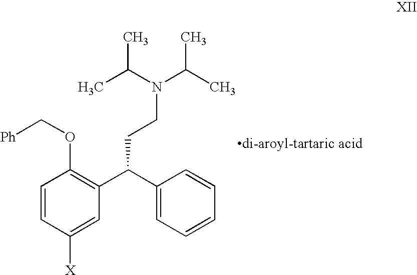
wherein ‘X’ is as defined for formula VII;
b) if required, separating the diastereomers of formula XII; and
c) neutralizing the product of step-(a) or separated diastereomers of step-(b) with a base in a suitable solvent to provide enantiomerically pure compound of formula VII.

25. The process of claim 24, wherein the optically active di-aroyl-tartaric acid used in step-(a) is selected from the group comprising (−)-di-p-toluoyl-L-tartaric acid, (+)-di-p-toluoyl-D-tartaric acid, (−)-dibenzoyl-L-tartaric acid, (+)-dibenzoyl-D-tartaric acid, and hydrates thereof; wherein the separation of the diastereomers in step-(b) is carried out by fractional crystallization; wherein the base used in step-(c) is an organic or inorganic base; and wherein the (R)—N,N-diisopropyl-3-(2-benzyloxy-5-halophenyl)-3-phenylpropylamine of formula VII or (R)—N,N-diisopropyl-3-(2-benzyloxy-5-bromophenyl)-3-phenylpropylamine of formula VII(i) (formula VII, wherein X is Br) obtained has enantiomeric purity of greater than about 98%.

26. The process of claim 25, wherein the optically active di-aroyl-tartaric acid is (−)-di-p-toluoyl-L-tartaric acid; wherein the organic base is selected from the group consisting of triethyl amine, dimethyl amine and tert-butyl amine; wherein the inorganic base is selected from the group consisting of sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide; and wherein the (R)—N,N-diisopropyl-3-(2-benzyloxy-5-halophenyl)-3-phenylpropylamine of formula VII or (R)—N,N-diisopropyl-3-(2-benzyloxy-5-bromophenyl)-3-phenylpropylamine of formula VII(i) has enantiomeric purity of greater than about 99%.

27. The process of claim 24, wherein the solvent used in step-(a) is selected from the group consisting of water, acetone, acetonitrile, methanol, ethanol, isopropyl alcohol, tert-butanol, dichloromethane, chloroform, carbon tetrachloride, dimethylformamide, dimethylsulphoxide, ethyl acetate, toluene, xylene, pentane, hexane, heptane, ethyl ether, isopropyl ether, tetrahydrofuran, 1,4-dioxane, ethyleneglycol, 1,2-dimethoxyethane, and mixtures thereof; wherein the solvent used for separation in step-(b) is selected from the group consisting of water, methanol, ethanol, isopropyl alcohol, propanol, tert-butyl alcohol, n-butanol, acetone, methyl ethyl ketone, methyl isobutyl ketone, diethyl ketone, ethyl acetate, methyl acetate, isopropyl acetate, tert-butyl methyl acetate, ethyl formate, acetonitrile, tetrahydrofuran, dimethylformamide, dimethylsulfoxide, dioxane, diethyl carbonate, and mixtures thereof; and wherein the solvent used in step-(c) is selected from the group comprising water, alcohols, ketones, cyclic ethers, aliphatic ethers, hydrocarbons, chlorinated hydrocarbons, nitriles, esters, and mixtures thereof.

28. The process of claim 27, wherein the solvent used in step-(a) is selected from the group consisting of methanol, ethanol, isopropyl alcohol, ethyl acetate, water and mixtures thereof; and wherein the solvent used in step-(c) is selected from the group consisting of water, methanol, ethanol, propanol, butanol, amyl alcohol, hexanol, acetone, methyl isobutyl ketone, tetrahydrofuran, dioxane, acetonitrile, ethyl acetate, isopropyl acetate, n-pentane, n-hexane and n-heptane, cyclohexane, toluene, xylene, methylene chloride, ethyl dichloride, chloroform and carbon tetrachloride, and mixtures thereof.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. Use of (R)—N,N-diisopropyl-3-(2-benzyloxy-5-halophenyl)-3-phenylpropylamine of formula VII or (R)—N,N-diisopropyl-3-(2-benzyloxy-5-bromophenyl)-3-phenylpropylamine of formula VII(i) obtained as per the process of claim 24 in the process for manufacture of fesoterodine or a pharmaceutically acceptable salt thereof.

42. (canceled)

43. A process for the preparation of a hydroxy compound of formula IV: 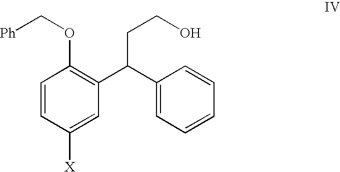
wherein ‘X’ represents a halogen atom selected from the group consisting of F, Cl, Br and I; comprising reducing the compound of formula III: 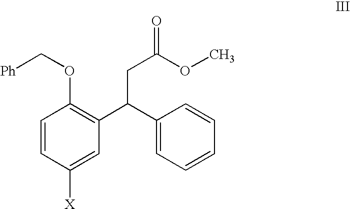
wherein ‘X’ is as defined for formula IV; with a suitable reducing agent in the presence of a Lewis acid.

44. A process for preparing a protected compound of formula V: 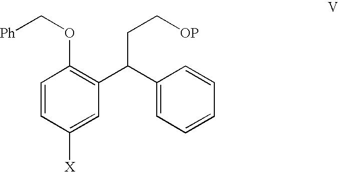
wherein ‘P’ represents a C₁-C₆-alkyl- or aryl-sulfonyl protecting group; and ‘X’ represents a halogen atom selected from the group consisting of F, Cl, Br and I; comprising reacting the compound of formula IV: 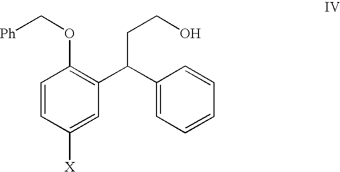
wherein ‘X’ is as defined for formula V; with a C₁-C₆-alkyl- or aryl-sulfonyl halide in the presence of an aliphatic organic base.

45. (canceled)

46. A process for preparing an ester compound of formula IX: 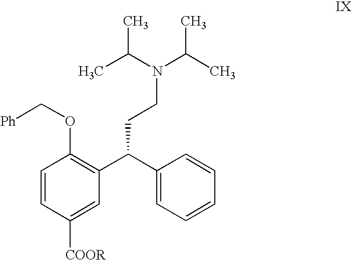
wherein ‘R’ represents C₁-C₆-alkyl-group such as methyl, ethyl and isopropyl; comprising esterifying (R)-4-benzyloxy-3-(3-diisopropylamino-1-phenylpropyl)-benzoic acid hydrochloride of formula VIII with a C₁-C₆-alcohol in the presence of acid chloride.

47. A process for preparing (R)-[4-benzyloxy-3-(3-diisopropylamino-1-phenylpropyl)-phenyl]-methanol of formula X: 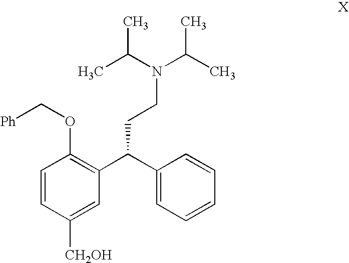
comprising reducing the compound of formula IX: 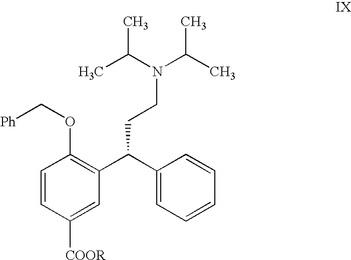
wherein ‘R’ represents C₁-C₆-alkyl-group such as methyl, ethyl and isopropyl; with a reducing agent in the presence of a Lewis acid.
